(19) United States
(12) Patent Application Publication
(10) Pub. No.: US 2022/0031967 A1
(43) Pub. Date: Feb. 3, 2022

(54) COVID-19 Respiratory THERAPY Device

(71) Applicant: Tae Yang Park, Seoul (KR)

(72) Inventor: Tae Yang Park, Seoul (KR)

(21) Appl. No.: 16/941,536

(22) Filed: Jul. 29, 2020

(51) Int. Cl. A61M 11/04; A61M 11/00; A61M 11/02

(52) U.S. Cl. CPC A61M 11/044; A61M 11/005; A61M 2205/3368; A61M 2202/206; A61M 2210/0618; A61M 11/02

(57) ABSTRACT

Provided is a COVID-19 respiratory therapy device, including: a main body (10) defining a predetermined interior space; a container (111) provided on an upper portion of the main body (10); an inhalation nozzle (50) installed on an upper portion of the container (111); a water tank (13) installed in the container (111) and having an open top; a vapor space defined by the container (111) and the inhalation nozzle (50); a vaporizer (20) installed in the water tank (13) and evaporating water in the water tank (13); a controller (30) provided in the interior space and controlling the vaporizer (20); and a discharge hole (53) provided on an upper portion of the inhalation nozzle (50).

### TECHNICAL FIELD

The present invention relates to a respiratory therapy device capable of treating a respiratory system by providing a therapeutic agent contained in evaporated water vapor that can be inhaled.

### TECHNICAL BACKGROUND

Recently, there are increasing cases of respiratory infection such as COVID-19. Respiratory diseases are caused by a virus that penetrates the human hotly through the respiratory system.

The reality is that, currently, there is no direct treatment for COVID-19, other than the administration of remedy.

### DETAILED DESCRIPTION

### Problems to be Solved

The present invention has been devised to solve the above-described problems. An object of the present invention is to provide a respiratory treatment device that directly delivers a therapeutic agent to the respiratory system of a patient with a respiratory disease when inhaled.

### Solution to Solve the Problem

In order to solve the above-mentioned problems, the present invention provides a respiratory therapy device capable of alleviating respiratory diseases by fumigating an antiviral herb and/or evaporating water containing a silver nano solution so that a patient can directly inhale. The respiratory treatment device includes a main body (10) and a inhalation nozzle (50).

The inhalation nozzle (50) is detachably installed on the main body (10). A container (111) is provided on an upper portion of the main body (10), and the inhalation nozzle (50) is installed on an upper portion of the container (111) portion.

The container (111) is provided with a water tank (13) for holding water. The water tank (13) is in the form of a bowl with an open top, and a vaporizer (20) is installed on an outer surface of the water rank (13).

The vaporizer (20) includes an ultrasonic vibrator (22) installed on a bottom of the water tank (13) and evaporates water in the water tank (13). The ultrasonic vibrator (22) allows the silver nanoparticles to be mixed into the vapor when the water containing the silver nanoparticles is evaporated. The vaporizer (20) includes a heater (21) installed on an outer surface of the water tank (13). The operation of the heater (21) is controlled by a controller (30).

The controller (30) may be implemented on a substrate installed in an interior space of the main body (10). In the water tank (13), a temperature sensor is installed to sense temperature of the water in the water tank (13). The controller (30) may control the operation of the heater (21) based on the temperature of the water measured by the temperature sensor.

The container (111) and the inhalation nozzle (50) define a vapor space that holds the water vapor which is evaporated by the vaporizer (20). In the vapor space, a medicine case capable of containing a natural medicine such as a herb is installed. The bottom, the side, and the top surfaces of the medicine case have pores through which a gas can flow.

When the water subject to evaporation contains the silver nano solution, the controller (30) operates the ultrasonic vibrator (22) and controls the water to be a first temperature. When the water subject to evaporation does not contain the silver nano solution, the controller (30) does not operate the ultrasonic vibrator (22) and controls the water to be a second temperature. The second temperature is higher than the first temperature.

### Advantages of Invention

According to the present invention, it is possible to treat a patient having a respiratory disease by supplying medicine in an inhale manner.

The respiratory disease may be caused by a virus. The medicine may have antiviral and sterilizing properties.

According to the present invention, with one treatment device, it is possible to incorporate different medicinal ingredients into the vapor.

According to the present invention, a configuration for incorporating different medicinal ingredients into steam or vapor can be used in common, thereby simplifying the product.

### BRIEF DESCRIPTION OF DRAWINGS

FIG. 1 is a perspective view of a main body (10) of the respiratory treatment device of the embodiment.

FIG. 2 is a perspective view of an inhalation nozzle of the respiratory treatment device of the embodiment.

FIG. 3 is an exploded perspective view of a medicine case of the respiratory treatment device of the embodiment.

FIG. 4 is a perspective view of a medicine case of the respiratory therapy device of the embodiment.

FIG. 5 is an exploded perspective view of the respiratory treatment device of the embodiment.

FIG. 6 is a cross-sectional view of a water tank (13) of the respiratory treatment device of the embodiment.

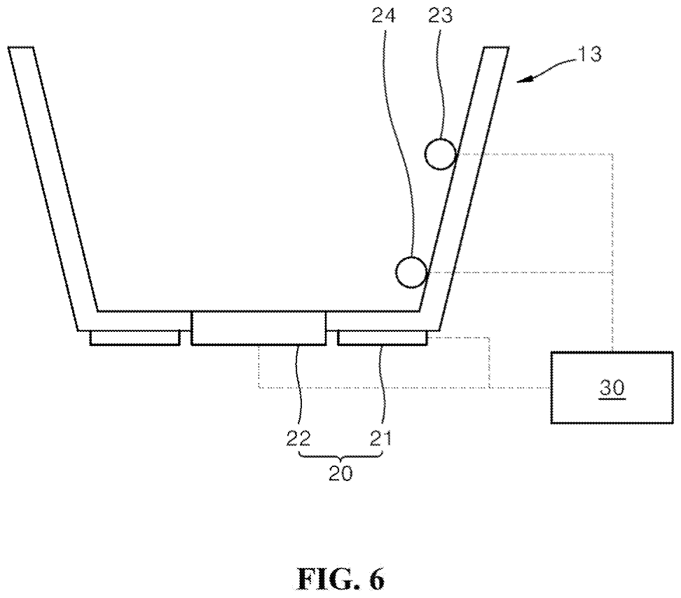

### EMBODIMENTS

Hereinafter, preferred embodiments of the present invention will be described in detail with reference to the accompanying drawings.

The respiratory therapy device of the embodiment includes a main body (10), a inhalation nozzle (50), and a medicine case (40). The inhalation nozzle (50) is installed on top of the main body (10). The medicine case (40) is provided in the vapor space defined by the inhalation nozzle (50).

Main Body (10)

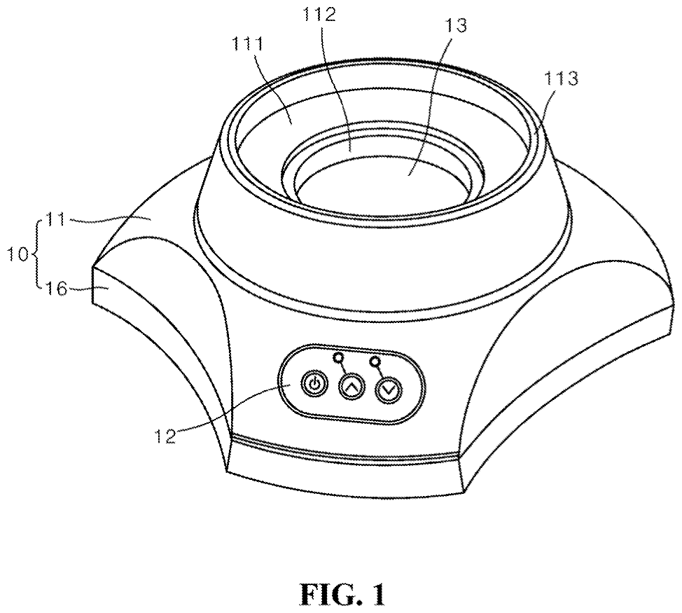

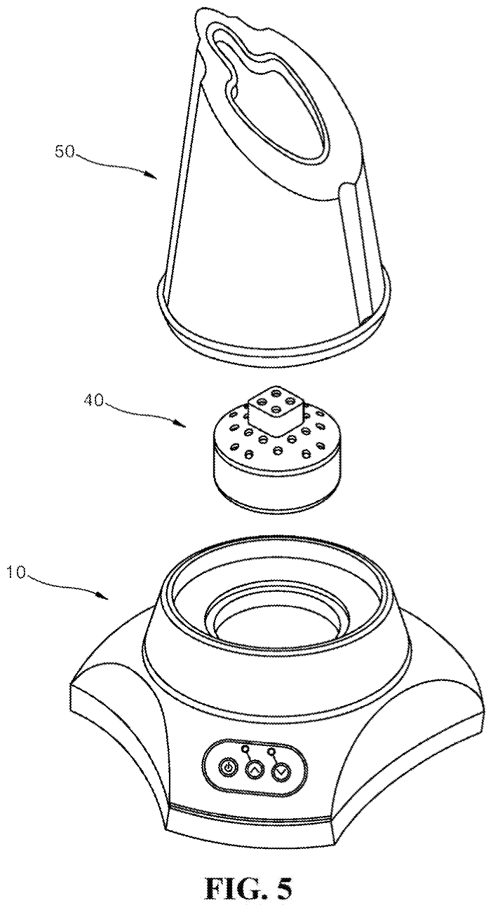

The main body (10) will be described with reference to FIGS. 1, 4, and 5. The main body (10) includes: a body housing (11) positioned on an upper portion; and a lower housing (16) coupled to a lower portion of the body housing (11).

The body housing (11) and the lower housing (16) are made of synthetic resin and are combined with each other to define a predetermined interior space. The input panel (12) is provided on a front surface of the body housing (11).

A user inputs commands for the treatment operation as well as the on/off of the respiratory treatment device through the input panel (12). The commands include a first command and a second command. The operation of the respiratory therapy device made by the first command is different from that made by the second command.

For example, when the first command is input, the device can operate under a first operating condition. When the second command is input, the device can operate under a second operating condition. That is, the commands can determine the operating conditions of the device.

A container (111) is provided on the body housing (111). The container (111) may have an open top. An upper end of the container (111) may extend upward. A circular hole (112) may be formed in a bottom center of the container (111). The circumference of the upper end extending upwards forms a body fitting part (113) that is detachably fastened with a inhalation nozzle (50), which will be described later. An inner space defined by the container (111) forms part of the vapor space to be described later.

A water tank (13) is installed in a hole (112) of the container (111). The water tank (13) may be in the form of a bowl with an open top. The water tank (13) ma be manufactured by drawing a metal plate that has high thermal conductivity. An upper end of the water tank (13) is connected and sealed to an inner circumferential surface of the hole (112). Thereby, the water tank (13) is integrated with the body housing (11). Accordingly, the water tank (13) also defines the interior space of the main body (10).

The water tank (13) accommodates water that generates steam that the patient can breathe and inhale. In order to evaporate water, a vaporizer (20) is installed in the water tank (13). Then, in the interior space of the main body (10), a controller (30) for controlling the vaporizer (20) is installed.

Inhalation Nozzle (50)

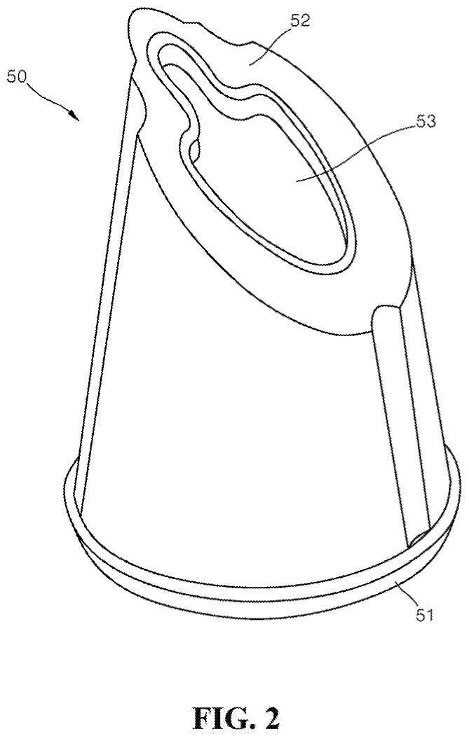

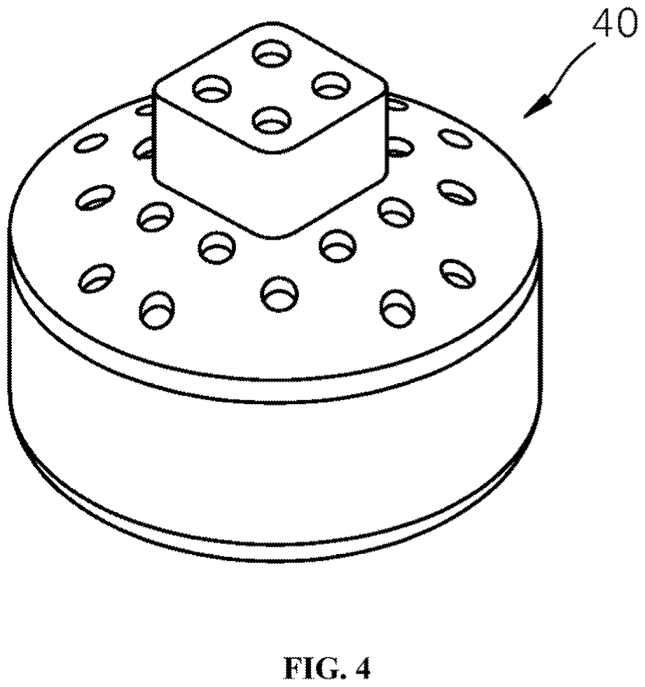

The inhalation nozzle (50) will be described with reference to FIGS. 2 and 4. The upper portion of the container (111) of the main body (10) is provided with a inhalation nozzle (50). The inhalation nozzle (50) may be made of a translucent or transparent synthetic resin. The inhalation nozzle (50) may be hollow. The bottom of the inhalation nozzle (50) may be open to the bottom. The inhalation nozzle (50) may be in a conical shape with an inclined surface (52) formed on the top.

The inclined surface (52) is provided with a discharge hole (53). The discharge hole (53) may be in a shape to which the nose or the mouth of a patient can be placed close. Therefore, the patient can breathe water vapor containing a therapeutic agent through the nose and also through the mouth. Since the discharge hole (53) is formed on the inclined surface (52), it is easier for the patient to bring the mouth or nose closer to the discharge hole (53).

The inhalation nozzle (50) is detachably mounted on the container (111). To this end, a nozzle fitting part (51) is provided around the lower end of the inhalation nozzle (50). The nozzle fitting part (51) has a shape complementary to the body fitting part (113). The nozzle fitting part (51) fits the body fitting part (113) so that the inhalation nozzle (50) can be mounted on the main body (10).

When the inhalation nozzle (50) is mounted on the main body (10), the interior space defined by the inhalation nozzle (50) and the container (111) becomes a vapor space. While discharging the inhalation nozzle (50) from the main body (10), the user may add a therapeutic agent to the container (111) and may supply water to the water tank (13). Additionally, when the inhalation nozzle (50) is mounted on the main body (10), it is possible to operate the respiratory therapy device.

Medicinal Case (40)

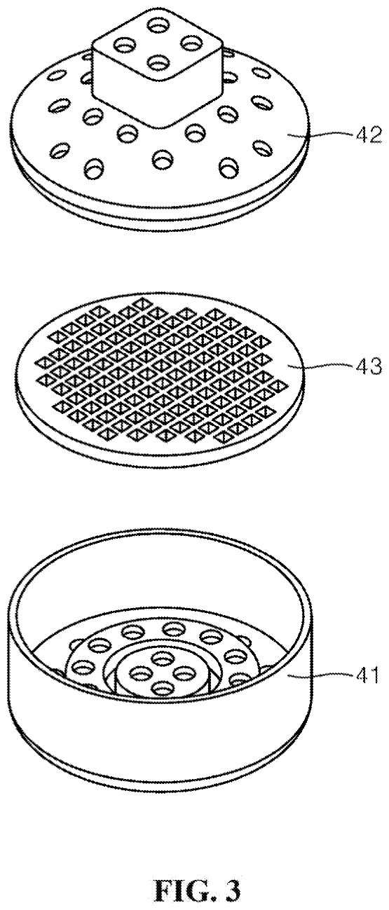

The medicine case (40) will be described with reference to FIGS. 3 and 4. The medicine case (40) may be in the form of a housing with multiple pores formed on the bottom and top surfaces. The therapeutic agent may be accommodated inside the medicine case (40).

To this end, the medicine case (40) can be configured so that its upper case (42)) is opened. Thus, the medicine case (40) may be made of a lower case (41) and an upper case (42). The lower case (41) forms a side wall and a bottom of the medicine case (40) and is opened to the top. The upper case (42) covers the top of the lower case (41). The upper portion of the upper case (42) is provided with a handle that protrudes upward. A number of pores are formed on the bottom surface of the lower case (41) and the upper case (42). Water vapor may pass through the pores.

A perforated plate (43) may be placed on the bottom of the lower Case (41). The perforated plate (43) is provided with pores smaller in size than the pores formed at the bottom of the lower case (41) so as to selectively allow water vapor to pass while preventing the treatment component from escaping downward.

The circumference of the lower end of the lower case (41) may be detachably fitted to the hole (112) of the container (111). Under such structure, the water vapor evaporated from the water tank (13) is forced to flow into the vapor space after passing through the inner space of the medicine case (40).

The medicine case (40) may be optionally installed in the container (111). That is, when the treatment is performed with only the treatment component that docs not use the medicine case, the medicine case (40) may not lie installed.

The medicine case (40), for example, may be made of a synthetic resin that does not elute components even at high temperatures. However, the material of the medicine case (40) is not limited to this.

Medicine

The medicine case (40) may contain a medicine. The medicine may contain a therapeutic agent for the respiratory system. The therapeutic agents contained in the medicine case (40) are mixed into steam (vaporized water) evaporated from the water tank (13). The warm vapor evaporated from the water tank (13) contains the therapeutic agent, and rises upward after passing through the medicine case (40).

The medicine may include antiviral natural herbs, such as herbs. However, the type of the medicine is not limited to herbs, and may be any medicine containing therapeutic agents, such as mugwort, that can soothe respiratory diseases.

In addition, a silver nano solution may be added to the respiratory treatment device. The silver nano solution is a solution in which silver nano particles are diluted. The silver nano solution may be added to the water in the water tank (13). Then, when the water evaporates from the water tank (13), the silver nanoparticles may evaporate together. It is normally difficult for the vapor that results from heating and vaporizing the water in the water tank (13) to contain silver nanoparticles. However, vaporizing the water using an ultrasonic vibrator (22) allows the vapor to contain very small amounts of silver nanoparticles.

Vaporizer

A vaporizer (20) may be installed in the water tank (13). The vaporizer (20) may include an ultrasonic vibrator (22) installed in the water rank (13). The ultrasonic vibrator (22) may be installed on the bottom of the water tank (13).

The ultrasonic vibrator (22) allows water vapor to contain silver nanoparticles when water is vaporized.

In addition, the vaporizer (20) may include a heater (21) installed in the water tank (13). The heater (21) may be installed on the bottom and/or side wall of the water tank (13). The heater (21) increases the temperature of the vapor inhaled into the respiratory tract, and also allows the water in the water tank (13) to boil and vaporize.

Control Part

The controller (30) may control the vaporizer (20). That is, the controller (30) can control the ultrasonic vibrator (22) and heater (21). The controller (30) is electrically connected to the ultrasonic vibrator (22) and the heater (21).

The water tank (13) includes: a first sensor (23) that can measure the temperature of the water inside the water tank (13); and a second sensor (24) that detects the silver nano component of the water contained in the water tank (13). The controller (30) is electrically connected to the first sensor (23) and the second sensor (24).

The controller (30) may control the ultrasonic vibrator (22) and heater (21) according to an operation command input by the user through the input panel (12). The controller (30) may control the ultrasonic vibrator (22) and heater (21) based on signals received from the first sensor (23) and the second sensor (24).

The respiratory treatment device may be used by mixing the sliver nano solution in the water tank (13), and alternatively may be used by putting the medicine in the medicine case (40). As for the medicine, only one or both of the silver nano solution or the medicine in the medicine case (40) can be used.

Regardless of whether or not the medicine is used, when the treatment is performed using the silver nano solution, the controller (30) may control the vaporizer (20) under the first operating condition. In addition, when the silver nano solution is not used and only the medicine in the medicine case (40) is used, or when the treatment is performed by simply evaporating the water in the water tank (13), the controller (30) can control the vaporizer (20) under the second operating condition.

When the first operation condition is input through the input panel (12), the controller (30) controls the vaporizer (20) according to the first operation condition. When a first signal, which indicates that the silver nano component is detected, is received from the second sensor (24), the controller (30) controls the vaporizer (20) according to the first operating condition.

When the second operation condition is input through the input panel (12), the controller (30) controls the vaporizer (20) according to the second operation condition. When the first signal is not received from the second sensor (24), that is, the silver nano component is not detected, the controller (30) controls the vaporizer (20) according to the second operating condition.

In the first operation condition, the controller (30) operates the sonic vibrator. Additionally, the controller (30) supplies a first output to the heater (21) and sets the temperature of the water in the water tank (13) at a first temperature. The temperature of the water in the water tank (13) is controlled based on the temperature of the water measured by the first sensor (23).

When only the ultrasonic vibrator (22) is operated, the patient may feel discomfort when inhaling, as the vapor particles are large and the temperature of the vapor is low. Therefore, a heater (21) is used together with the ultrasonic vibrator (22) to increase the temperature of the steam.

In the second operation condition, the controller (30) does not operate the ultrasonic vibrator (22). Additionally, the controller (30) supplies a second output to the heater (21) and sets the temperature of the water in the water tank (13) to a second temperature. The second output is greater than the first output. The second temperature is higher than the first temperature. The second temperature may correspond to the boiling point of water. Therefore, when proceeding with the treatment without silver nanoparticles, the patient can inhale very warm vapors into the respiratory tract.

According to the above-described invention, the respiratory treatment device can perform two different treatment methods. In addition, since the heater (21) is applicable in both operating conditions, different treatment conditions can be elicited using one heater (21). Thus, the device can be simplified. In addition, it is possible to treat a patient with a respiratory disease caused by a virus by supplying in are inhale manner a medicine with antiviral and sterilizing agents.

### REFERENCE NUMERALS

10: main body-interior space
11: body housing
111: container
112: hole
113: body fitting part
12: input panel
13: water tank
16: lower housing
20: vaporizer
21: heater
22: Ultrasonic vibrator
23: first sensor
24: second sensor
30: controller
40: medicinal case
41: lower case
42: upper case
43: perforated plate
50: inhalation nozzle
51: nozzle fitting part
52 inclined surface
53: discharge hole

What is claimed is:

1. A COVID-19 respiratory therapy device, comprising:
a main body (10) defining an interior space;
a container (111) provided on an upper portion of the main body (10);
an inhalation nozzle (50) installed on an upper portion of the container (111);
a water tank (13) installed in the container (111) and having an open top;
a vapor space defined by the container (111) and the inhalation nozzle (50);
a vaporizer (20) installed in the water tank (13) and evaporating water in the water tank (13);
a controller (30) provided in the interior space and controlling the vaporizer (20); and
a discharge hole (53) provided on an upper portion of the inhalation nozzle (50).

2. The COVID-19 respiratory therapy device of claim 1,
wherein the container (111) is provided with a hole (112) at a center,
wherein the water tank (13) is installed in the hole (112).

3. The COVID-19 respiratory therapy device of claim 1,
wherein the inhalation nozzle (50) is installed detachably to the container (111),
wherein a nozzle fitting part (51) is provided around a lower end of the inhalation nozzle (50),
wherein a body fitting part (113) is in such a shape as complementary to the nozzle fitting part (51) and is provided around an upper end of the container (111).

4. The COVID-19 respiratory therapy device of claim 1,
wherein the inhalation nozzle (50) is provided with an inclined surface (52),
wherein the inclined surface (52) is provided with a discharge hole (53) and configured for accommodating a user's nose.

5. The COVID-19 respiratory therapy device of claim 1,
wherein the vaporizer (20) includes a heater (21),
wherein the heater (21) is installed on a bottom or art side wall of the water tank (13).

6. The COVID-19 respiratory therapy device of claim 5,
wherein the water tank (13) is provided with a first sensor (23),
wherein the first sensor (23) senses temperature of the water in the water tank (13), and
wherein the controller (30) is electrically connected to the first sensor (23).

7. The COVID-19 respiratory therapy device of claim 1, further comprising:
a medicine case (40) installed in the vapor space,
wherein the medicine case (40) includes a porous box.

8. The COVID-19 respiratory therapy device of claim 7,
wherein the vaporizer (20) is configured to accommodate a therapeutic agent,
wherein the therapeutic agent includes an antiviral herb.

9. The COVID-19 respiratory therapy device of claim 1,
wherein the water tank (13) is provided with a first sensor (23),
wherein the first sensor (23) senses temperature of the water in the water tank (13), and
wherein the controller (30) is electrically connected to the first sensor (23).

10. The COVID-19 respiratory therapy device of claim 1,
wherein the vaporizer (20) includes an ultrasonic vibrator (22),
wherein the ultrasonic vibrator (22) is installed on a bottom of the water tank (13).

11. The COVID-19 respiratory therapy device of claim 10,
wherein the vaporizer (20) is configured of accommodating a therapeutic agent,
wherein the therapeutic agent includes a silver nano solution mixed in the water tank (13).

12. The COVID-19 respiratory therapy device of claim 11,
wherein the water tank (13) is provided with a second sensor (24),
wherein die second sensor (24) detects a silver nano component contained in the water in the water tank (13),
wherein the controller (30) is electrically connected to the second sensor (24).

13. The COVID-19 respiratory therapy device of claim 12,
wherein the controller (30) operates the ultrasonic vibrator (22) when the controller (30) receives a first signal from the second sensor (24),
wherein the first signal is generated when the second sensor (24) detects the silver nano component.

14. The COVID-19 respiratory therapy device of claim 13,
wherein the vaporizer (20) comprises a heater (21),
wherein the heater (21) heats the water in the water tank (13),
wherein the controller (30) sets the water in the water tank (13) to a first temperature when the controller (30) receives the first signal from the second sensor (24),
wherein the controller (30) sets the water in the water tank (13) to a second temperature when the controller (30) does not receive the first signal from the second sensor (24),
wherein the first temperature is lower than the second temperature.

15. The COVID-19 respiratory therapy device of claim 13,
wherein the vaporizer (20) comprises a heater (21),
wherein the heater (21) heats the water in the water tank (13),
wherein the controller (30) transmits a first output to the heater when the controller (30) receives the first signal from the second sensor (24),
wherein the controller (30) transmits a second output to the heater (21) when the controller (30) does not receive the first signal from the second sensor (24),
wherein the first output is lower than the second output.

16. The COVID-19 respiratory therapy device of claim 10,
wherein the main body (10) is provided with an input panel (12),
wherein the input panel (12) inputs a first operating condition and a second operating condition,
wherein the vaporizer (20) comprises a heater (21),
wherein the heater (21) heats the water in the water tank (13),
wherein, when the first operating condition is input through the input panel (12), the controller (30) operates the ultrasonic vibrator (22) and heats the water in the water tank (13) to a first temperature,
wherein, when the second operating condition is input through the input panel (12), the controller (30) does not operate the ultrasonic vibrator (22) and heats the water in the water tank (13) to a second temperature,
wherein the first: temperature is lower than the second temperature.
